(19) United States
(12) Patent Application Publication
(10) Pub. No.: US 2017/0151139 A1
(43) Pub. Date: Jun. 1, 2017

(54) POWERFUL HAIR TREATMENT AGENT WITH ANTI-WASHOUT EFFECT

(71) Applicant: Henkel AG & Co. KGaA, Duesseldorf (DE)

(72) Inventors: Volker Scheunemann, Lueneburg (DE); Erik Schulze zur Wiesche, Hamburg (DE)

(73) Assignee: Henkel AG & Co. KGaA, Duesseldorf (DE)

(21) Appl. No.: 15/363,821

(22) Filed: Nov. 29, 2016

(30) Foreign Application Priority Data
Dec. 1, 2015 (DE) 10 2015 223 852.0

(51) Int. Cl. A61K 8/34; A61K 8/42; A61K 8/73; A61K 8/36; A61Q 17/00; A61K 8/362; A61K 8/92; A61Q 5/02; A61Q 5/00; A61K 8/46; A61K 8/365

(52) U.S. Cl. CPC A61K 8/347; A61K 8/463; A61K 8/42; A61K 8/731; A61K 8/36; A61K 8/365; A61K 8/362; A61K 8/922; A61K 8/737; A61Q 5/02; A61Q 5/004; A61Q 17/005; A61K 2800/884; A61K 2800/594; A61K 2800/596; A61K 2800/5426

(57) ABSTRACT

Hair treatment agents containing at least one anionic surfactant, at least one amphoteric and/or non-ionic surfactant, at least one cationic polysaccharide polymer, o-phenylphenol and/or salts of o-phenylphenol, at least one organic acid and at least one lipid component reduce or prevent the washout of color from dyed hair.

### CROSS-REFERENCE TO RELATED APPLICATION

This application claims priority to German Patent Application No. 10 2015 223 852.0, filed Dec. 1, 2015, which is incorporated herein by reference in its entirety.

### TECHNICAL FIELD

The invention relates to hair treatment agents, in particular shampoos and what are known as conditioners, comprising an active substance combination for gentle and effective care of the hair.

### BACKGROUND

Not least due to heavy stressing of the hair, for example by dyeing or perming as well as by cleaning of the hair with shampoos and by environmental pollution, the importance of care products having an effect that lasts as long as possible is increasing. Care products of this type influence the natural structure and the properties of the hair. By way of example, the wet and dry combability of the hair, the hold and volume of the hair can be optimized following such care treatments, or the hair can be protected against increased splitting.

It has therefore long been customary to subject hair to a special post-treatment. Here, the hair is treated with special active substances, for example quaternary ammonium salts or special polymers, usually in the form of a conditioner. As a result of this treatment, depending on the formulation, the combability, the hold and the volume of the hair are improved and the amount of split ends are reduced.

Multi-functional cosmetic products are known in the prior art as well. These include, in particular, what are known as “2-in-1” shampoos, which not only clean the hair, but also condition it. Products of this type are held in high esteem by consumers because, due to their product performance, they eradicate the need for at least one process step, for example conditioning with a conventional hair conditioner.

Products for changing the natural hair color also play a prominent role in hair cosmetics. A distinction is made between permanent, semi-permanent, or temporary dyeing systems, these being based on chemical and/or natural dyes. The hair colors produced artificially by permanent, semi-permanent or temporary dyes, however, have the disadvantage that they can change undesirably, for example during or after cleaning of the hair.

The term “undesirable change” is understood to mean the fading or bleeding and also the loss of the color brilliance of the coloring of the hair attained by the respective dyeing. Ambient influences and/or the effects of the sun can intensify these changes further.

The use of bivalent metal salts in hair dyes to improve the durability and therefore fastness of the coloration is known from EP 2438900 A1.

There is still a need to provide active substances or active substance combinations for hair treatment agents having good nourishing properties which in addition increase the adhesion of dyes to the hair fibers and thus preserve the fastness of the artificially produced hair color, and also to further develop hair treatment agents in this respect.

It has been found, however, that the water hardness can sometimes negatively influence the nourishing properties of hair treatment agents, and therefore the nourishing power of the same product can be either too low or too high depending on water quality.

A nourishing power that is too high or “over-nourishment” (occurrence of what is known as a build-up effect) is understood to mean in particular a greasy hair feel, lack of hair volume, and/or a lank, unkempt appearance of the hair, whereas damaged hair (broken and dull hair that is prone to splitting) is an indicator of too little nourishing power.

### BRIEF SUMMARY

Hair treatment agents and methods are provided herein. In an embodiment, a hair treatment agent includes:
a) at least one anionic surfactant,
b) at least one amphoteric and/or non-ionic surfactant,
c) at least one cationic polysaccharide polymer,
d) o-phenylphenol and/or salts of o-phenylphenol

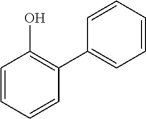 [SMILES: Oc1ccccc1-c1ccccc1]

e) at least one organic acid, and
a) at least one lipid component.

In another embodiment, a method for treating hair includes applying a hair treatment agent to dry or moist hair, wherein the hair treatment agent includes:
a) at least one anionic surfactant,
b) at least one amphoteric and/or non-ionic surfactant,
c) at least one cationic polysaccharide polymer,
d) o-phenylphenol and/or salts of o-phenylphenol

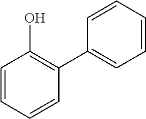 [SMILES: Oc1ccccc1-c1ccccc1]

e) at least one organic acid, and
f) at least one lipid component,

where the hair treatment is agent is left on the hair for a time period of about 30 to about 300 seconds, and then rinsed out thereafter.

### DETAILED DESCRIPTION

The following detailed description is merely exemplary in nature and is not intended to limit the hair treatment agents and methods for treating hair. Furthermore, there is no intention to be bound by any theory presented in the preceding background or the following detailed description.

The object of the present application was therefore to provide nourishing hair treatment agents which are compatible with the skin and which have excellent foaming properties and which can be rinsed out well with water and additionally reduce or prevent the washout of color from dyed hair.

The nourishing hair treatment agents should have a uniform conditioning power irrespective of the water quality and in particular should not weigh down fine hair and/or damaged hair, but should strengthen the structure of the hair and thus protect it against splitting and breaking and should improve combability and the feel of the hair. In addition, said agents should have a germicidal effect.

It has now been found that a combination of certain ingredients has a particularly positive effect on dyed hair and the hair follicle treated with said ingredients.

In one embodiment, hair treatment agents include:
a) at least one anionic surfactant,
b) at least one amphoteric and/or non-ionic surfactant,
c) at least one cationic polysaccharide polymer,
d) o-phenylphenol and/or salts of o-phenylphenol

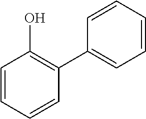 [SMILES: Oc1ccccc1-c1ccccc1]

e) at least one organic acid,
f) at least one lipid component.

Hair treatment agents in accordance with embodiments are, for example, hair shampoos, hair conditioners, conditioning shampoos, hairsprays, hair rinse-out treatments, hair deep treatments, hair masks, hair tonics, permanent wave fixing solutions, hair-coloring shampoos, hair dyes, hair fixers, hair setting agents, hairstyling preparations, blow-dry lotions, foam fixers, hair gels, hair waxes, or combinations thereof. In view of the fact that men often avoid the application of a number of different agents and/or a number of application steps, agents that can be readily applied by men are preferred. Preferred agents are therefore shampoos, conditioning agents or hair tonics.

The hair treatment agents contain at least one anionic surfactant.

All anionic surface-active substances suitable for use on the human body are suitable as anionic surfactants and emulsifiers for the compositions as contemplated herein. These substances are characterized by a water-solubilizing anionic group, such as a carboxylate, sulfate, sulfonate or phosphate group and a lipophilic alkyl group having approximately 8 to 30 C atoms. In addition, glycol or polyglycol ether groups, ester, ether and amide groups, and also hydroxyl groups can be contained. Examples of suitable anionic surfactants and emulsifiers, in each case in the form of the sodium, potassium and ammonium salts as well as the mono-, di- and trialkanol ammonium salts having 2 to 4 C atoms in the alkanol group, are
linear and branched fatty acids having 8 to 30 C atoms (soaps),
ether carboxylic acids of formula R—O—(CH₂—CH₂O)ₓ—CH₂—COOH, in which R is a linear alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
linear alkane sulfonates having 8 to 24 C atoms,
linear alpha-olefin sulfonates having 8 to 24 C atoms,
alpha sulfo fatty acid methyl esters of fatty acids having 8 to 30 C atoms,
acyl glutamates of formula (T-I),

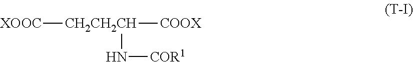

in which R¹CO stands for a linear or branched acyl group having 6 to 22 carbon atoms and 0, 1, 2 or 3 double bonds and X stands for hydrogen, an alkali metal and/or alkaline earth metal, ammonium, alkylammonium, alkanolammonium or glucammonium, for example acylglutamates deriving from fatty acids having 6 to 22, preferably 12 to 18 carbon atoms, such as C_{12/14} or C_{12/18} coconut fatty acid, lauric acid, myristic acid, palmitic acid and/or stearic acid, in particular sodium-N-cocoyl glutamate and sodium-N-stearoyl-L-glutamate,
esters of a hydroxyl-substituted di- or tricarboxylic acid of general formula (T-II),

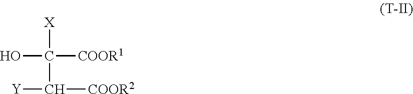

in which X=H or is a —CH₂COOR-Gruppe, Y=H or is —OH under the condition that Y=H if X=—CH₂COOR, R, R¹ und R² independently of one another denote a hydrogen atom, an alkali or alkaline earth metal cation, an ammonium group, the cation of an organoammonium base or a group Z deriving from a polyhydroxylated organic compound selected from the group of etherified (C₆-C₁₈) alkyl polysaccharides having 1 to 6 monomeric saccharide units and/or the etherified aliphatic (C₆-C₁₆) hydroxyalkyl polyols having 2 to 16 hydroxyl groups, with the proviso that at least one of the groups R, R¹ or R² is a group Z,
esters of sulfosuccinic acid or sulfosuccinates of the general formula (T-III),

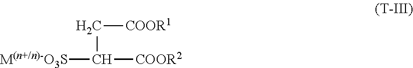

in which M^{(n+/n)} for n=1 is a hydrogen atom, an alkali metal cation, an ammonium group or the cation of an organoammonium base, and for n=2 is an alkaline earth metal cation, and R¹ and R² independently of one another mean a hydrogen atom, an alkali or alkaline earth metal cation, an ammonium group, the cation of an organoammonium base or a group Z deriving from a polyhydroxylated organic compound selected from the group of etherified (C₆-C₁₈) alkyl polysaccharides having 1 to 6 monomeric saccharide units and/or the etherified aliphatic (C₆-C₁₆) hydroxyalkyl polyols having 2 to 16 hydroxyl groups, with the proviso that at least one of the groups R, R¹ or R² is a group Z,
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
alkyl sulfates and alkyl polyglycol ether sulfates of the formula R—(O—CH₂—CH₂)ₓ—OSO₃H, in which R is a preferably linear alkyl group having 8 to 30 C atoms and x=0 or 1 to 12,
mixed surface-active hydroxyl sulfonates according to DE-A-37 25 030,
esters of tartaric acid and citric acid with alcohols which are addition products of approximately 2 to 15 molecules of ethylene oxide and/or propylene oxide with C₈₋₂₂ fatty alcohols,
alkyl and/or alkenyl ether phosphates,
sulfated fatty acid alkylene glycol esters,
monoglyceride sulfates and monoglyceride ether sulfates.

Preferred anionic surfactants are acyl glutamates, acyl isethionates, acyl sarcosinates and acyl taurates, each having a linear or branched acyl group with 6 to 22 carbon atoms and 0, 1, 2 or 3 double bonds, which in particularly preferred embodiments is selected from an octanoyl, decanoyl, lauroyl, myristoyl, palmitoyl and stearoyl group, esters of tartaric acid, citric acid or succinic acid or salts of these acids with alkylated glucose, in particular products with the INCI name Disodium Coco-Glucoside Citrate, Sodium Coco-Glucoside Tartrate and Disodium Coco-Glucoside Sulfosuccinate, alkyl polyglycol ether sulfates and ether carboxylic acids having 8 to 18 C atoms in the alkyl group and up to 12 ethoxy groups in the molecule, sulfosuccinic acid mono- and dialkyl esters having 8 to 18 C atoms in the alkyl group and sulfosuccinic acid monoalkyl polyoxyethyl esters having 8 to 18 C atoms in the alkyl group and 1 to 6 ethoxy groups.

Anionic surfactants that are more preferred are alkyl sulfates, alkyl polyglycol ether sulfates, and ether carboxylic acid salts having 10 to 18 C atoms in the alkyl group and up to 12 glycol ether groups in the molecule and sulfosuccinic acid mono esters and dialkyl esters having 8 to 18 C atoms in the alkyl group and sulfosuccinic acid mono-alkyl polyoxy ethyl esters having 8 to 18 C atoms in the alkyl group and 1 to 6 oxyethyl groups.

Particularly preferred anionic surfactants are the alkali or ammonium salts of lauryl ether sulfate with a degree of ethoxylation of from 2 to 4 EO.

Preferred hair treatment agents contain—in relation to their weight—about 0.5 to about 20% by weight, preferably about 0.75 to about 15% by weight, more preferably about 1 to about 12% by weight, and in particular about 2 to about 10% by weight of anionic surfactant(s).

Particularly preferred hair treatment agents as contemplated herein are characterized in that they contain—in relation to their weight—about 0.5 to about 20% by weight, preferably about 0.75 to about 15% by weight, more preferably about 1 to about 12% by weight, and in particular about 2 to about 10% by weight of alkyl(ether)sulfates of general formula R—(OCH₂—CH₂)ₙ—OSO₃X, in which R is a straight-chained or branched, saturated or unsaturated alkyl group having 8 to 24 C atoms, n is the number 0 or 1 to 12, and X means an alkali, earth alkaline, ammonium or alkanol anion.

The hair treatment agents as contemplated herein contain at least one amphoteric surfactant and/or at least one non-ionic surfactant. Surfactants that have both a negatively and a positively charged functional group are referred to as amphoteric surfactants or as zwitterionic surfactants.

Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing in each case 8 to 18 carbon atoms in the alkyl or acyl group and cocoacylaminoethyl hydroxyethyl carboxymethyl glycinate. The fatty acid amide derivative known under the INCI name of Cocamidopropyl Betaine is preferred.

Further examples of suitable ampholytic surfactants are N-alkyl glycines, N-alkyl aminopropionic acids, N-alkyl aminobutyric acids, N-alkyl iminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 24 carbon atoms in the alkyl group. Particularly preferred ampholytic surfactants are N-cocoalkylaminopropionate, cocoacylaminoethyl aminopropionate and C₁₂-C₁₈ acyl sarcosine.

Preferred hair treatment agents as contemplated herein contain—in relation to their weight—about 0.3 to about 10% by weight, preferably about 0.5 to about 8% by weight, more preferably about 0.75 to about 6% by weight, and in particular about 1 to about 5% by weight of amphoteric surfactant(s).

Particularly preferred hair treatment agents as contemplated herein are characterized in that they contain amphoteric surfactant(s) from the groups of
N-alkyl glycines,
N-alkyl propionic acids,
N-alkyl aminobutyric acids,
N-alkyl iminodipropionic acids,
N-hydroxyethyl-N-alkylamidopropyl glycines,
N-alkyl taurines,
N-alkyl sarcosines,
2-alkyl aminopropionic acids having in each case around 8 to 24 C atoms in the alkyl group,
alkyl aminoacetic acids having in each case around 8 to 24 C atoms in the alkyl group,
N-cocoalkyl aminopropionate,
cocoacylaminoethyl aminopropionate,
C₁₂-C₁₈ acyl sarcosine,
N-alkyl-N,N-dimethylammonium glycinates, for example cocoalkyl dimethylammonium glycinate,
N-acylaminopropyl-N,N-dimethylammonium glycinates, for example cocoacylaminopropyl dimethylammonium glycinate,
2-alkyl-3-carboxymethyl-3-hydroxyethyl imidazolines each with 8 to 18 C atoms in the alkyl or acyl group,
cocoacylaminoethyl hydroxyethyl carboxymethyl glycinate,
the compounds known by the INCI name Cocamidopropyl Betaine, and
the compounds known by the INCI name Disodium Cocoamphodiacetate,
   wherein preferred agents contain the amphoteric surfactant(s) in amounts of from about 0.3 to about 10% by weight, preferably about 0.5 to about 8% by weight, more preferably about 0.75 to about 6% by weight, and in particular about 1 to about 5% by weight, in each case in relation to the total agent.

Particularly preferred hair treatment agents contain, as amphoteric surfactants, betaines of the formula (Bet-I)

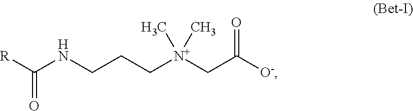

in which R stands for a straight-chained or branched, saturated or mono- or polyunsaturated alkyl or alkenyl group having 8 to 24 carbon atoms.

These surfactants are referred to according to the INCI nomenclature as Amidopropyl Betaines, the representatives derived from coconut fatty acids being preferred and referred to as Cocamidopropyl Betaines. Surfactants of formula (Bet-I) that are used with particular preference as contemplated herein are a mixture of the following representatives:

H₃C—(CH₂)₇—C(O)—NH—(CH₂)₃N⁺(CH₃)₂CH₂CH₂COO⁻
H₃C—(CH₂)₉—C(O)—NH—(CH₂)₃N⁺(CH₃)₂CH₂COO⁻
H₃C—(CH₂)₁₁—C(O)—NH—(CH₂)₃N⁺(CH₃)₂CH₂COO⁻
H₃C—(CH₂)₁₃—C(O)—NH—(CH₂)₃N⁺(CH₃)₂CH₂COO⁻
H₃C—(CH₂)₁₅—C(O)—NH—(CH₂)₃N⁺(CH₃)₂CH₂COO⁻
H₃C—(CH₂)₇—CH═CH—(CH₂)₇—C(O)—NH—(CH₂)₃N⁺(CH₃)₂CH₂COO⁻

Surfactants of formula (Bet-I) are particularly preferably used within narrower quantity ranges. Here, preferred hair treatment agents as contemplated herein are those that contain—in relation to their weight—about 0.25 to about 8% by weight, more preferably about 0.5 to about 7% by weight, more preferably about 0.75 to about 6.5% by weight and in particular about 1 to about 5.5% by weight of surfactant(s) of the formula (Bet-I).

In addition to the amphoteric surfactants of formula (Bet-I) or instead of these, the hair treatment agents as contemplated herein can particularly preferably contain, as amphoteric surfactants, betaines of formula (Bet-II)

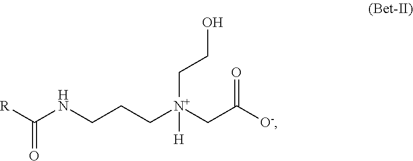

in which R stands for a straight-chained or branched, saturated or mono- or polyunsaturated alkyl or alkenyl group having 8 to 24 carbon atoms.

These surfactants are referred to according to the INCI nomenclature as Amphoacetates, wherein the representatives deriving from coconut fatty acids are preferred and are referred to as Cocoamphoacetates.

For reasons relating to their manufacture, surfactants of this type always also contain betaines of formula (Bet-IIa)

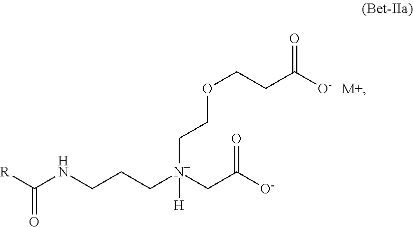

in which R stands for a straight-chained or branched, saturated or mono- or polyunsaturated alkyl or alkenyl group having 8 to 24 carbon atoms and M stands for a cation.

These surfactants are referred to according to the INCI nomenclature as Amphodiacetates, wherein the representatives deriving from coconut fatty acids are preferred and are referred to as Cocoamphodiacetates.

Surfactants of formula (Bet-II) that are used with particular preference as contemplated herein are a mixture of the following representatives:

H₃C—(CH₂)₇—C(O)—NH—(CH₂)₂N⁺(CH₂CH₂OH)CH₂CH₂COO⁻
H₃C—(CH₂)₉—C(O)—NH—(CH₂)₂N⁺(CH₂CH₂OH)CH₂CH₂COO⁻
H₃C—(CH₂)₁₁—C(O)—NH—(CH₂)₂N⁺(CH₂CH₂OH)CH₂CH₂COO⁻
H₃C—(CH₂)₁₃—C(O)—NH—(CH₂)₂N⁺(CH₂CH₂OH)CH₂CH₂COO⁻
H₃C—(CH₂)₁₅—C(O)—NH—(CH₂)₂N⁺(CH₂CH₂OH)CH₂CH₂COO⁻
H₃C—(CH₂)₇—CH═CH—(CH₂)₇—C(O)—NH—(CH₂)₂N⁺(CH₂CH₂OH)CH₂CH₂COO⁻

Surfactants of formula (Bet-II) are particularly preferably used within narrower quantity ranges. Here, preferred hair treatment agents as contemplated herein are those that contain—in relation to their weight—about 0.25 to about 8% by weight, more preferably about 0.5 to about 7% by weight, more preferably about 0.75 to about 6.5% by weight, and in particular about 1 to about 5.5% by weight of surfactant(s) of formula (Bet-II).

To summarize, preferred cosmetic agents as contemplated herein are those in which the group R in the formulas (Bet-I) and (Bet-II) is selected from H₃C—(CH₂)₇—H₃C—(CH₂)₉—H₃C—(CH₂)₁₁—H₃C—(CH₂)₁₃—H₃C—(CH₂)₁₅—H₃C—(CH₂)₇—CH═CH—(CH₂)₇— or mixtures thereof.

The hair treatment agents can contain non-ionic surfactant(s). If they are free from amphoteric surfactants, they necessarily contain a non-ionic surfactant.

Suitable non-ionic surfactants include, for example
addition products of 4 to 30 mol ethylene oxide and/or 0 to 5 mol propylene oxide with linear fatty alcohols having 8 to 22 C atoms, with fatty acids having 12 to 22 C atoms, and with alkyl phenols having 8 to 15 C atoms in the alkyl group,
ethylene oxide and polyglycerol addition products with methylglucoside fatty acid esters, fatty acid alkanolamides, and fatty acid glucamides,
C₈-C₃₀ fatty acid mono- and di-esters of addition products of 1 to about 30 mol ethylene oxide with glycerol,
amine oxides,
sorbitol fatty acid esters and addition products of ethylene oxide with sorbitol fatty acid esters, such as polysorbates,
fatty acid alkanol amides of the following general formula,

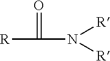

in which R preferably means a linear or branched, saturated or unsaturated alkyl or alkenyl group having 8 to 24 carbon atoms and the groups R′ stand for hydrogen or for the group —(CH₂)ₙOH, in which n means the numbers 2 or 3, with the proviso that at least one of the groups R′ stands for the aforementioned group —(CH₂)ₙOH,
sugar fatty acid esters and addition products of ethylene oxide with sugar fatty acid esters,
addition products of ethylene oxide with fatty acid alkanol amides and fatty amines, and/or
alkyl (oligo)glucosides,
mixtures of alkyl (oligo)glucosides and fatty alcohols, for example the commercially available product Montanov® 68,
addition products of 5 to about 60 mol ethylene oxide with castor oil and hardened castor oil,
partial esters of polyols having 3-6 carbon atoms with saturated fatty acids having 8 to 22 C atoms,
sterols. Sterols are understood to mean a group of steroids which carry a hydroxyl group on C atom 3 of the steroid backbone and are isolated both from animal tissue (zoosterols) and from plant fats (phytosterols). Examples of zoosterols are cholesterol and lanosterol. Examples of suitable phytosterols are ergosterol, stigmasterol, and sitosterol. Sterols are also isolated from fungi and yeasts and are known in this case as mycosterols.
phospholipids. These include, in particular, the glucose phospholipids, which are obtained for example as lecithins or phosphatidylcholines from, for example, egg yolk or plant seeds (for example soybeans).

Suitable alkyl (oligo)glycosides can be selected from compounds of the general formula RO-[G]ₓ, in which [G] preferably derives from aldoses and/or ketoses having 5-6 carbon atoms, preferably from glucose.

The index number x stands for the degree of oligomerization (DP), i.e. for the distribution of the mono- and oligoglycosides. The index number x preferably has a value in the range from 1 to 10, particularly preferably in the range from 1 to 3, wherein it is not necessarily an integer, but can be a fractional number, which can be determined analytically.

Particularly preferred alkyl (oligo)glycosides have a degree of oligomerization between 1.2 and 1.5.

The group R preferably stands for at least one alkyl and/or alkenyl group having 4 to 24 C atoms.

Particularly preferred alkyl (oligo)glycosides are the compounds known by the INCI names Caprylyl/Capryl Glucoside, Decyl Glucoside, Lauryl Glucoside and Coco Glucoside.

Suitable amine oxides can be selected from at least one compound of general formulas (A-I) or (A-II)

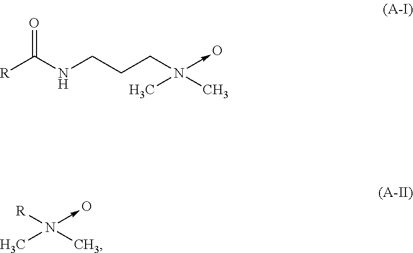

in which R stands in each case for a straight-chained or branched, saturated or mono- or polyunsaturated alkyl or alkenyl group having 6 to 24 carbon atoms, preferably having 8 to 18 carbon atoms.

The surfactants of the above-mentioned formulas (A-I) or (A-II) known by the INCI names Cocamine Oxide, Lauramine Oxide and/or Cocamidopropylamine Oxide and commercially available from various providers are particularly preferred.

Suitable C₈-C₃₀ fatty acid mono- and di-esters of addition products of 1 to about 30 mol ethylene oxide with glycerol are preferably understood to be those that are known by the INCI names PEG(1-10) Glyceryl Cocoate, in particular PEG-7 Glyceryl Cocoate.

It can also be advantageous to combine the ethoxylated fatty acid esters with further ethoxylated fatty acid esters. Such product mixtures are commercially available—for example under the name “Antil 200®” (INCI name: PEG-200 Hydrogenated Glyceryl Palmate, PEG-7 Glyceryl Cocoate) from the company Evonik.

Particularly preferred non-ionic surfactants which can be contained in the hair treatment agents as contemplated herein are fatty acid alkanol amides, in particular the compounds known by the INCI names Cocamide MEA and/or Cocamide MIPA; Alkyl (oligo)glucoside, in particular the compounds known by the INCI names Caprylyl/Capryl Glucoside, Decyl Glucoside, Lauryl Glucoside and/or Coco Glucoside; C₈-C₃₀ fatty acid mono- and di-esters of addition products of 1 to about 30 mol ethylene oxide with glycerol, in particular the compound known by the INCI name PEG-7 Glyceryl Cocoate; and/or addition products of 4 to about 30 mol ethylene oxide and/or 0 to 5 mol propylene oxide with linear fatty alcohols having 8 to 22 C atoms.

Cocamide MEA and/or PEG-7 Glyceryl Cocoate are particularly preferred on account of their foam-stabilizing and regreasing properties.

Hair treatment agents that are preferred as contemplated herein contain—in relation to their weight—about 0.3 to about 10% by weight, preferably about 0.5 to about 8% by weight, more preferably about 0.75 to about 6% by weight, and in particular about 1 to about 5% by weight of non-ionic surfactant(s).

The hair treatment agents can contain cationic surfactant(s). Cationic surfactants that can be used as contemplated herein are those of the following type: quaternary ammonium compounds, esterquats, and amidoamines. Preferred quaternary ammonium compounds are ammonium halides, in particular chlorides and bromides, such as alkyltrimethylammonium chlorides, dialkyldimethylammonium chlorides, and trialkylmethylammonium chlorides. The long alkyl chains of these surfactants preferably have 10 to 18 carbon atoms, for example as in cetyl trimethyl ammonium chloride, stearyl trimethyl ammonium chloride, distearyl dimethyl ammonium chloride, lauryl dimethyl ammonium chloride, lauryl dimethyl benzyl ammonium chloride, and tricetyl methyl ammonium chloride. Further preferred cationic surfactants are the imidazolium compounds known by the INCI names quaternium-27 and quaternium-83.

Particularly preferred hair treatment agents as contemplated herein are characterized in that they contain, as cationic nourishing substance—in relation to their weight—about 0.05 to about 7.5% by weight, preferably about 0.1 to about 5% by weight, particularly preferably about 0.2 to about 3.5% by weight and in particular about 0.25 to about 2.5% by weight of cationic surfactant(s) from the group of quaternary ammonium compounds and/or esterquats and/or amidoamines, wherein preferred cationic surfactant(s) is/are selected from
alkyltrimethylammonium chlorides having preferably 10 to 18 carbon atoms in the alkyl group and/or
dialkyldimethylammonium chlorides having preferably 10 to 18 carbon atoms in the alkyl group and/or
trialkylmethylammonium chlorides having preferably 10 to 18 carbon atoms in the alkyl group and/or
cetyl trimethyl ammonium chloride and/or
stearyl trimethyl ammonium chloride and/or
distearyl dimethyl ammonium chloride and/or
lauryl dimethyl ammonium chloride and/or
lauryl dimethyl benzyl ammonium chloride and/or
tricetyl methyl ammonium chloride
quaternium-27 and/or
quaternium-83 and/or
N-methyl-N(2-hydroxyethyl)-N,N-(ditalgacyloxyethyl)ammonium methosulfate and/or
N-methyl-N(2-hydroxyethyl)-N,N-(distearoyloxyethyl)ammonium methosulfate and/or
N,N-dimethyl-N,N-distearoyloxyethyl ammonium chloride and/or
N,N-di-(2-hydroxyethyl)-N,N-(fatty acid ester ethyl) ammonium chloride.

The hair treatment agents can contain at least one cationic polysaccharide polymer.

Cationic polysaccharide polymers increase the nourishing ability of the hair treatment agents as contemplated herein (in particular the efficacy of the agents as contemplated herein with respect to hair breakage). Suitable cationic polysaccharide polymers can be selected from cationic cellulose compounds and/or from cationic guar derivatives.

Particularly preferred hair treatment agents as contemplated herein contain, as cationic polysaccharide polymer(s) c)—in relation to the weight of the agent—about 0.01 to about 3% by weight, preferably about 0.05 to about 2% by weight, more preferably about 0.1 to about 1.5% by weight, and in particular about 0.15 to about 0.8% by weight of at least one polymer from the group of cationic cellulose polymers and/or cationic guar derivatives.

In embodiments, cationic cellulose compounds are those that carry more than one permanent cationic charge in at least one side chain. Cellulose is constructed from beta-1,4-glycosidically linked D-glucopyranose units and forms unbranched, water-insoluble chains. A “side chain” of a cellulose is defined by chemical substituents which bond to the cellulose structure and do not include only native cellulose, since they have been introduced subsequently, for example by chemical synthesis.

Quaternized cellulose polymers originating from hydroxy(C₂-C₄)alkyl celluloses, particularly preferably from hydroxyethyl celluloses, are preferred.

Such polymers are known to a person skilled in the art and are commercially available from various companies. The cationic cellulose derivatives known under the INCI names polyquaternium-4, polyquaternium-10, polyquaternium-24, polyquaternium-67 and/or polyquaternium-72 are particularly preferred. Polyquaternium-10, polyquaternium-24 and/or polyquaternium-67 are very particularly preferred, and polyquaternium-10 is in particular preferred.

Preferred hair treatment agents as contemplated herein contain, as cationic polysaccharide polymer(s) c)—in relation to the weight of the agent—about 0.01 to about 3% by weight, preferably about 0.05 to about 2% by weight, more preferably about 0.1 to about 1.5% by weight, and in particular about 0.15 to about 0.8% by weight of at least one polymer from the group of polyquaternium-4, polyquaternium-10, polyquaternium-24, polyquaternium-67 and/or polyquaternium-72.

Particularly preferred hair treatment agents as contemplated herein contain, as cationic polysaccharide polymer(s) c)—in relation to the weight of the agent—about 0.01 to about 3% by weight, preferably about 0.05 to about 2% by weight, more preferably about 0.1 to about 1.5% by weight, and in particular about 0.15 to about 0.8% by weight of polyquaternium-10.

In embodiments, suitable cationic guar derivatives are cationic hydroxyalkyl guar derivatives, preferably cationic hydroxyethyl trimethylammonium guar and/or cationic hydroxypropyl trimethylammonium guar having average molecular weights between about 100,000 and about 2,000,000 daltons. The cationic guar polymers known under the INCI name Guar Hydroxypropyltrimonium Chlorides having a molecular weight (weight average) between about 200,000 and about 1,600,000 daltons are particularly preferred. The cationic charge density of these guar polymers is preferably at least about 0.4 meq/g, preferably at least about 0.5 meq/g, and in particular at least about 0.6 meq/g. Their nitrogen content lies preferably in a range of from about 1.1 to about 1.8% by weight (in relation to their total weight).

Cationic guar derivatives known under the INCI name Guar Hydroxypropyltrimonium Chlorides are known to a person skilled in the art and are obtainable for example under the trade names Cosmedia® Guar, N-Hance® and/or Jaguar® from various providers.

Particularly preferred hair treatment agents as contemplated herein contain, as cationic polysaccharide polymer(s) c)—in relation to the weight of the agent—about 0.01 to about 3% by weight, preferably about 0.05 to about 2% by weight, more preferably about 0.1 to about 1.5% by weight, and in particular about 0.15 to about 0.8% by weight of Guar Hydroxypropyltrimonium Chlorides.

The hair treatment agents contain o-phenylphenol and/or salts of o-phenylphenol. What is preferably used is o-phenylphenol, wherein the use is particularly effective within certain narrower quantity ranges.

Preferred hair treatment agents as contemplated herein contain—in relation to their weight—about 0.001 to about 0.4% by weight, preferably about 0.01 to about 0.3% by weight, more preferably about 0.025 to about 0.25% by weight, and in particular about 0.05 to about 0.2% by weight of o-phenylphenol.

In addition to o-phenylphenol or instead of this, salts of o-phenylphenol can also be used. Here, the alkali metal salts sodium orthophenylphenolate, potassium orthophenylphenolate and ammonium orthophenylphenolate, magnesium orthophenylphenolate, calcium phenylphenolate, zinc orthophenylphenolate, iron orthophenylphenolate and manganese orthophenylphenolate have proven to be particularly suitable, inter alia.

Preferred hair treatment agents as contemplated herein contain salt(s) from the group sodium orthophenylphenolate, potassium orthophenylphenolate and ammonium orthophenylphenolate, magnesium orthophenylphenolate, calcium phenylphenolate, zinc orthophenylphenolate, iron orthophenylphenolate and manganese orthophenylphenolate in a total amount of from about 0.001 to about 0.4% by weight, preferably about 0.01 to about 0.3% by weight, more preferably about 0.025 to about 0.25% by weight, and in particular about 0.05 to about 0.2% by weight, in each case in relation to the weight of the agent.

Very particularly preferred hair treatment agents contain—in relation to their weight—about 0.001 to about 0.4% by weight, preferably about 0.01 to about 0.3% by weight, more preferably about 0.025 to about 0.25% by weight, and in particular about 0.05 to about 0.2% by weight of sodium orthophenylphenolate.

The hair treatment agents contain at least one organic acid.

Typical representatives for organic acids are aliphatic mono- and dicarboxylic acids, such as acetic acid, propionic acid, oxalic acid and 1,3-propanedioic acid, and also aromatic carboxylic acids, such as benzoic acid. Further organic acids are, for example, hydroxycarboxylic acids, such as glycolic acid, citric acid, tartaric acid, malic acid and lactic acid. Unsaturated mono- or dicarboxylic acids such as fumaric acid or α-ketocarboxylic acids, such as pyruvic acid (2-oxopropanoic acid) are also organic acids as contemplated herein.

Hair treatment agents that are preferred as contemplated herein contain—in relation to their weight—about 0.001 to about 10% by weight, preferably about 0.01 to about 5% by weight, more preferably about 0.05 to about 3% by weight, and in particular about 0.1 to about 2% by weight of organic acid(s).

Particularly preferred hair treatment agents contain—in relation to their weight—about 0.001 to about 10% by weight, preferably about 0.01 to about 5% by weight, more preferably about 0.05 to about 3% by weight, and in particular about 0.1 to about 2% by weight, of organic acid(s) from the group of formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, bicarbaminic acid, 4,4′-dicyano-6,6′-binicotinic acid, 8-carbamoyloctanoic acid, 1,2,4-pentanetricarboxylic acid, 2-pyrrole carboxylic acid, 1,2,4,6,7-napthalene pentaacetic acid, malonaldehydic acid, 4-hydroxyphthalamidic acid, 1-pyrazole carboxylic acid, gallic acid or propane tricarboxylic acid, glycolic acid, lactic acid, malic acid, tartaric acid or citric acid.

Some of the above-mentioned acids are particularly preferred because they also provide the hairs treated using the agents as contemplated herein with a pleasing glossy appearance. Extremely preferred hair treatment agents contain—in relation to their weight—about 0.001 to about 10% by weight, preferably about 0.01 to about 5% by weight, more preferably about 0.05 to about 3% by weight, and in particular about 0.1 to about 2% by weight of organic acid(s) from the group of formic acid, oxalic acid, maleic acid, lactic acid, tartaric acid or citric acid.

The hair treatment agents contain at least one lipid component.

All fats and fat-like substances which preferably melt in a range of about 30-150° C. can be used as contemplated herein as lipid components. These include, inter alia, fats (triglycerides), mono- and diglycerides, waxes, fatty and wax alcohols, fatty acids, esters and/or ethers of fatty alcohols and also fatty acid amides or any mixtures of these substances.

Fats are understood to mean triacylglycerols, i.e. the triple esters of fatty acids with glycerol. Preferred triacylglycerols are those as lipid component with a melting point between about 30° and about 150° C. and containing saturated, unbranched and unsubstituted fatty acid esters. They can also be mixed esters, i.e. triple esters formed from glycerol with different fatty acids. What are known as hardened fats and oils, obtained by partial hydrogenation, can also be used as contemplated herein and are particularly well suited as consistency enhancers. Plant hardened fats and oils are preferred, for example hardened castor oil, peanut oil, soybean oil, colza oil, rapeseed oil, cottonseed oil, soybean oil, sunflower oil, palm oil, palm kernel oil, linseed oil, almond oil, corn oil, olive oil, sesame oil, cocoa butter and coconut fat.

In a preferred embodiment the lipid component is selected from the group of triple esters of glycerol with fatty acids. The triple esters of glycerol with C₁₈-C₆₀ fatty acids and in particular C₁₈-C₃₆ fatty acids are particularly preferred. These include hardened castor oil, a triple ester formed from glycerol and a hydroxystearic acid, which for example is commercially available under the name Cutina® HR, and glycerol tristearate, glycerol tribehenate (for example Syncrowax® HRC), glycerol tripalmitate, or the triglycerol mixtures known under the name Syncrowax® HGLC.

A combination of hardened castor oil and C₁₈-C₃₆ fatty acid triglycerides (for example Syncrowax® HGLC) is also preferred as contemplated herein. The storage stability is increased as a result of the combination of these triglycerides. A combination of hardened castor oil and long-chain C₂₀-C₄₀ fatty acids (for example Performacid® 350 Acid) has also proven to be advantageous as lipid component.

Besides the triglycerides, it is also possible to use mono- and diglycerides or mixtures of the glycerides as lipid components. The glyceride mixtures preferred as contemplated herein include the combination of Cutina® HR (hardened castor oil) and Novata® AB (mixture of C₁₂-C₁₈ mono-, di- and triglycerides).

Fatty alcohols that can be used as contemplated herein include, for example, the unbranched C₁₄-C₅₀ fatty alcohols, in particular the C₁₄-C₃₀ fatty alcohols recovered from natural fats, oils and waxes, such as myristyl alcohol, 1-pentadecanol, cetyl alcohol, 1-heptadecanol, stearyl alcohol, 1-nonadecanol, arachidyl alcohol, 1-heneicosanol, behenyl alcohol, brassidyl alcohol, lignoceryl alcohol, ceryl alcohol or myricyl alcohol. Unbranched, saturated and unsubstituted fatty alcohols are preferred as contemplated herein.

However, branched, saturated or unsaturated fatty alcohols can also be used as contemplated herein. Fatty alcohol cuts, as occur with the reduction of naturally occurring fats and oils, can also be used as contemplated herein.

C₁₂-C₄₀ fatty acids or mixtures thereof are also suitable as lipid components. These include, for example, lauric, tridecanoic, myristic, pentadecanoic, palmitic, margaric, stearic, nonadecanoic, arachinic, behenic, lignocerinic, cerotinic, melissic, erucic and elaeostearic acid and also substituted fatty acids, such as 12-hydroxystearic acid, and the amides or monoethanolamides of fatty acids, wherein this list is exemplary and is in no way limiting. Among the fatty acids, a C₂₀-C₄₀ fatty acid mixture commercially available under the name Performacid® 350 Acid is preferably suitable.

Waxes are understood to mean natural or artificially obtained substances having the following properties: they have a firm to set hard consistency, are rough to finely crystalline, color-translucent to opaque, and melt above about 30° C. without decomposition. They already have a low viscosity just above the melting point and are not stringy and demonstrate a heavily temperature-dependent consistency and solubility. For example, natural plant waxes having a melting point of about 30-150° C. can be used as contemplated herein, such as candelilla wax, carnauba wax, Japan wax, esparto grass wax, cork wax, guaruma wax, rice seed oil wax, sugar beet wax, ouricuri wax, montan wax, sunflower wax, fruit waxes such as orange wax, lemon wax, grapefruit wax, bay wax (=bay berry wax), and animal waxes, such as beeswax, shellac wax, spermaceti, lanolin, and rump fat. In embodiments, tt can also be advantageous to use hydrogenated or hardened waxes. Natural waxes that can be used as contemplated herein also include mineral waxes, such as ceresin and ozokerite, or petrochemical waxes, such as petrolatum, paraffin wax, and microwax. Chemically modified wax, in particular hard wax, such as montan ester wax, sasol wax, and hydrogenated jojoba wax, can also be used as wax component. The synthetic waxes that can be used as contemplated herein include, for example, wax-like polyalkylene wax and polyethylene glycol wax.

The wax component can also be selected from the group of esters of saturated and/or unsaturated, branched and/or unbranched alkane carboxylic acids and saturated and/or unsaturated, branched and/or unbranched alcohols, from the group of esters of aromatic carboxylic acids, dicarboxylic acids, tricarboxylic acids and hydroxycarboxylic acids (for example 12-hydroxystearic acid) and saturated and/or unsaturated, branched and/or unbranched alcohols, and also from the group of lactides of long-chain hydroxycarboxylic acids, provided the wax component or the totality of wax components melts in a range of about 30-150° C. These include, for example, C₁₆-C₄₀-alkyl stearates, C₂₀-C₄₀ alkyl stearates (for example Kester Wax@ K82H), C₂₀-C₄₀ dialkyl esters of dimer acids, C₁₈-C₃₈ alkyl hydroxy stearoyl stearates or C₂₀-C₄₀ alkyl erucates. In addition, C₃₀-C₅₀ alkyl beeswax, tristearyl citrate, triisostearyl citrate, stearyl heptanoate, stearyl octanoate, trilauryl citrate, ethylene glycol dipalmitate, ethylene glycol distearate, ethylene glycol di(12-hydroxystearate), stearyl stearate, palmityl stearate, stearyl behenate, cetyl ester, cetearyl behenate and behenyl behenate can be used advantageously. Silicone wax might also be advantageous.

Regardless of the type of lipid component(s) used, preferred hair treatment agents as contemplated herein are those that contain—in relation to their weight—about 0.01 to about 5% by weight, preferably about 0.05 to about 3% by weight, more preferably about 0.1 to about 2.5, and in particular about 0.2 to about 2% by weight of lipid component(s).

In preferred hair treatment agents, the lipid component(s) is/are selected from triglyceride fats, mono- and diglycerides of saturated, linear C₁₂-C₄₀ fatty acids, natural and synthetic waxes, fatty and wax alcohols, C₁₀-C₄₀ fatty acids, esters of fatty alcohols, and fatty acids not liquid at 25° C., and also fatty acid amides and any mixtures of these substances.

Particularly preferred hair treatment agents as contemplated herein contain—in relation to their weight—about 0.01 to about 5% by weight, preferably about 0.05 to about 3% by weight, more preferably about 0.1 to about 2.5 and in particular about 0.2 to about 2% by weight of triglyceride fats and/or mono- and diglycerides of saturated, linear C₁₂-C₄₀ fatty acids and/or natural and synthetic waxes and/or fatty and wax alcohols and/or C₁₀-C₄₀ fatty acids and/or esters of fatty alcohols and fatty acids not liquid at 25° C. and/or fatty acid amides or any mixtures of these substance.

The hair treatment agents contain the above-described active substances preferably in a cosmetically acceptable carrier. In embodiments, an aqueous or aqueous-alcoholic carrier is employed.

The cosmetic carrier preferably contains at least about 50% by weight, more preferably at least about 60% by weight, particularly preferably at least about 70% by weight, and in particular preferably at least about 75% by weight of water.

Furthermore, the cosmetic carrier can contain about 0.01 to about 40% by weight, preferably about 0.05 to about 30% by weight, and in particular about 0.1 to about 20% by weight of at least one alcohol.

Suitable alcohols include, for example, ethanol, ethyldiglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol or mixtures of these alcohols.

The water-soluble alcohols are particularly preferred. Ethanol, 1,2-propylene glycol, glycerol, benzyl alcohol and mixtures of these alcohols are preferred in particular.

For very good skin (scalp) compatibility of the hair treatment agents as contemplated herein, it is advantageous if said agents have a slightly acidic pH value.

It has been found that the agents as contemplated herein have particularly good skin compatibility and gentleness in a pH range of from 4.2 to 5.8.

In a first preferred embodiment the hair treatment agents as contemplated herein therefore preferably have a pH value in a range of from 4.2 to 5.8, more preferably of from 4.25 to 5.6, particularly preferably of from 4.3 to 5.5, extremely preferably of from 4.35 to 5.4, and in particular preferably of from 4.4 to 5.3.

The hair treatment agents as contemplated herein can contain plant oils, plant butters, and/or plant waxes. These plant oil components provide the hair with improved combability and manageability and increase the shine of the hair.

Suitable plant oil components include natural (plant) oils and/or butters, which typically contain triglycerides and mixtures of triglycerides.

Preferred natural oils are coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, lady's smock oil, thistle oil, macadamia nut oil, grape seed oil, amaranth seed oil, bamboo oil, olive oil, wheatgerm oil, pumpkin seed oil, mallow oil, hazelnut oil, safflower oil, canola oil, sasanqua oil, jojoba oil, rambutan oil, cocoa butter and/or shea butter.

Carnauba wax, beeswax and/or candelilla wax can be used with preference as suitable natural or plant wax.

Particularly preferred plant oil components are (sweet) almond oil, peach kernel oil, apricot kernel oil, amaranth seed oil, argan oil, olive oil, jojoba oil, cocoa butter and/or shea butter.

Apricot kernel oil, argan oil, olive oil and/or jojoba oil are particularly preferred.

In a preferred embodiment the hair treatment agents as contemplated herein preferably contain coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, lady's smock oil, thistle oil, macadamia nut oil, grape seed oil, amaranth seed oil, bamboo oil, olive oil, wheatgerm oil, pumpkin seed oil, mallow oil, hazelnut oil, safflower oil, canola oil, sasanqua oil, jojoba oil, rambutan oil, cocoa butter and/or shea butter.

Within this embodiment it is particularly preferred if the hair treatment agents as contemplated herein contain (sweet) almond oil, peach kernel oil, apricot kernel oil, amaranth seed oil, argan oil, olive oil, jojoba oil, cocoa butter and/or shea butter.

The weight share of the at least one plant oil, the plant butter and/or the plant wax in the total weight of the hair treatment agent as contemplated herein is preferably about 0.02 to about 2.50% by weight, more preferably from about 0.03 to about 2.00% by weight, particularly preferably about 0.04 to about 1.50% by weight, and in particular about 0.05 to about 1.00% by weight.

Besides the aforementioned essential and optional constituents, the hair treatment agents as contemplated herein, in a further preferred embodiment for further increasing the nourishing properties of the agents, can contain at least one further hair-conditioning active substance, which can be selected from the group of
protein hydrolyzates,
vitamins,
plant extracts and/or
glycerol.

Suitable protein hydrolyzates are to be understood to be product mixtures that can be obtained by acid-, base- or enzyme-catalyzed degradation of proteins. Protein hydrolyzates of plant, animal and/or marine origin can be used.

Animal protein hydrolyzates are, for example, elastin, collagen, keratin, silk and milk protein hydrolyzates, which can also be present in the form of salts. Such products are marketed for example under the trade names Dehylan® (Cognis), Promois® (Interorgana) Collapuron® (Cognis), Nutrilan® (Cognis), Gelita-Sol® (German gelatin factories Stoess & Co), Lexein® (Inolex) and Kerasol® (Croda).

Protein hydrolyzates of plant origin are preferred, for example soy, almond, rice, pea, potato and wheat protein hydrolyzates. Such products are obtainable, for example, under the trade names Gluadin® (Cognis), DiaMin® (Diamalt), Lexenin® (Inolex) and Crotein® (Croda).

In addition, cationized protein hydrolyzates can be used, wherein the basic protein hydrolyzate can originate from animals, for example, from collagen, milk or keratin, from plants, for example, from wheat, maize, rice, potatoes, soya or almonds, from marine life, for example, from fish collagen or algae, or from biotechnologically obtained protein hydrolyzates. The protein hydrolyzates forming the basis of the cationic derivatives can be obtained from the corresponding proteins by a chemical hydrolysis, particularly alkaline or acid hydrolysis, by an enzymatic hydrolysis and/or a combination of both types of hydrolysis. The hydrolysis of proteins generally produces a protein hydrolyzate with a molecular weight distribution from about 100 daltons up to several thousand daltons. Cationic protein hydrolyzates that are preferred are those of which the base protein content has a molecular weight of about 100 to about 25,000 daltons, preferably about 250 to about 5,000 daltons. Moreover, cationic protein hydrolyzates are understood to include quaternized amino acids and their mixtures. Quaternization of the protein hydrolyzates or the amino acids is often carried out using quaternary ammonium salts such as, for example, N,N-dimethyl-N-(n-alkyl)-N-(2-hydroxy-3-chloro-n-propyl) ammonium halides. Moreover, the cationic protein hydrolyzates can also be further derivatized. Typical examples of cationic protein hydrolyzates and derivatives are the commercially available products known under the INCI names Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyl Hydrolyzed Plant Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Plant Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, and Quaternium-79 Hydrolyzed Wheat Protein.

Regardless of the source (plant, animal, marine etc.), protein hydrolyzates contain individual amino acids, oligopeptides and optionally polypeptides depending on the degree of hydrolysis.

The hair treatment agents as contemplated herein preferably contain at least one oligopeptide which comprises at least one amino acid sequence Glu-Glu-Glu

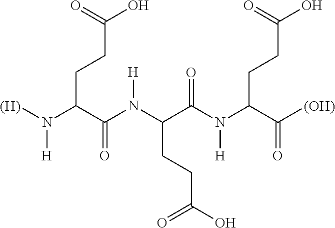

wherein the amino group can be free or protonated and the carboxyl group can be free or deprotonated.

Preferred hair treatment agents as contemplated herein are characterized in that they contain—in relation to their weight—about 0.0001 to about 10% by weight of at least one oligopeptide which comprises at least one amino acid sequence Glu-Glu-Glu

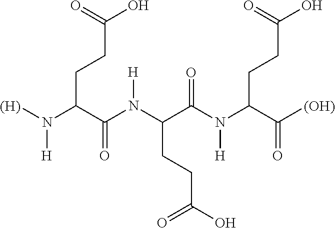

wherein the amino group can be free or protonated and the carboxyl group can be free or deprotonated.

In these formulas, as in all preceding formulas, the bracketed hydrogen atom of the amino group and also the bracketed hydroxyl group of the acid function means that the groups in question can be present as such (then, it is an oligopeptide having the relevant number of amino acids as illustrated (in formula 3 above)), or that the amino acid sequence is present in an oligopeptide which also comprises further amino acids—depending on where the further amino acid(s) is/are bonded, the bracketed constituents of the above-mentioned formula are replaced by the further amino acid group(s).

These preferred hair treatment agents as contemplated herein contain—in relation to their weight—about 0.0001 to about 10% by weight of at least one oligopeptide which comprises at least one amino acid sequence Glu-Glu-Glu, i.e. at least three successive glutaminic acids.

Oligopeptides in the sense of the present application are condensation products of amino acids linked in an acid amide-like manner by peptide bonds and comprising at least 3 and at most 25 amino acids.

In preferred hair treatment agents as contemplated herein the oligopeptide comprises 5 to 15 amino acids, preferably 6 to 13 amino acids, particularly preferably 7 to 12 amino acids, and in particular 8, 9 or 10 amino acids.

Depending on whether further amino acids are bonded to the sequence Glu-Glu-Glu and depending on the type of these amino acids, the molar mass of the oligopeptide contained in the agents as contemplated herein can vary. Hair treatment agents used with preference as contemplated herein are characterized in that the oligopeptide has a molar mass of from about 650 to about 3000 daltons, preferably from about 750 to about 2500 daltons, particularly preferably from about 850 to about 2000 daltons, and in particular from about 1000 to about 1600 daltons.

To summarize, preferred hair treatment agents are characterized in that the oligopeptide comprises 5 to 15 amino acids, preferably 6 to 13 amino acids, particularly preferably 7 to 12 amino acids, and in particular 8, 9 or 10 amino acids, and has a molar mass of from about 650 to about 3000 daltons, preferably from about 750 to about 2500 daltons, particularly preferably from about 850 to about 2000 daltons, and in particular from about 1000 to about 1600 daltons.

As is clear from the preferred number of amino acids in the oligopeptides and the preferred molar mass range, oligopeptides which do not consist solely of the three glutaminic acids, but which comprise further amino acids bonded to this sequence are preferably used. These further amino acids are preferably selected from certain amino acids, whereas certain other representatives are less preferred as contemplated herein.

It is thus preferred when the oligopeptides used in the agents as contemplated herein do not contain any methionine.

It is more preferred when the oligopeptides used in the agents as contemplated herein do not contain any cysteine and/or cystine.

It is more preferred when the oligopeptides used in the agents as contemplated herein do not contain any aspartic acid and/or asparagine.

It is more preferred when the oligopeptides used in the agents as contemplated herein do not contain any serine and/or threonine.

By contrast, it is preferred when the oligopeptides used in the agents as contemplated herein contain tyrosine.

It is more preferred when the oligopeptides used in the agents as contemplated herein contain leucine.

It is more preferred when the oligopeptides used in the agents as contemplated herein contain isoleucine.

It is more preferred when the oligopeptides used in the agents as contemplated herein contain arginine.

It is more preferred when the oligopeptides used in the agents as contemplated herein contain valine.

Particularly preferred oligopeptides or amino acid sequences contained in the preferred oligopeptides will be described below:

A particularly preferred oligopeptide additionally contains tyrosine, which is preferably bonded via its acid function to the Glu-Glu-Glu sequence. Hair treatment agents that are preferred as contemplated herein are therefore characterized in that the oligopeptide contained therein comprises at least one amino acid sequence Tyr-Glu-Glu-Glu

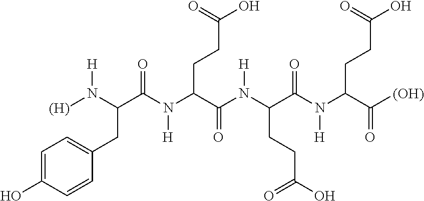

wherein the amino group can be free or protonated and the carboxyl group can be free or deprotonated.

A further particularly preferred oligopeptide additionally contain isoleucine, which is preferably bonded via its amino acid function to the Glu-Glu-Glu sequence. Hair treatment agents that are preferred as contemplated herein are therefore characterized in that the oligopeptide contained therein comprises at least one amino acid sequence Glu-Glu-Glu-Ile

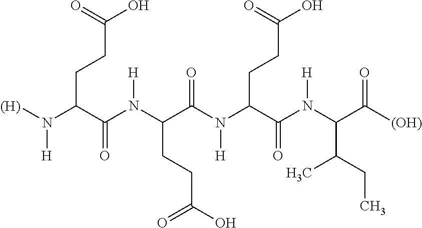

wherein the amino group can be free or protonated and the carboxyl group can be free or deprotonated.

Oligopeptides that comprise both aforementioned amino acids (tyrosine and isoleucine) are preferred as contemplated herein. Here, hair treatment agents as contemplated herein that are particularly preferred are those in which the oligopeptide contained in the hair treatment agent comprises at least one amino acid sequence Tyr-Glu-Glu-Glu-Ile

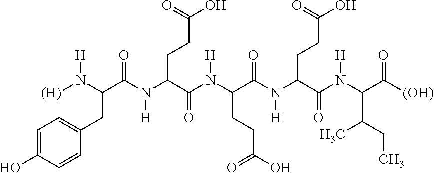

wherein the amino group can be free or protonated and the carboxyl group can be free or deprotonated.

Oligopeptides that are more preferred additionally contain arginine which is preferably bonded to isoleucine.

Here, hair treatment agents as contemplated herein that are particularly preferred are those in which the oligopeptide contained in the hair treatment agent comprises at least one amino acid sequence Tyr-Glu-Glu-Glu-Ile-Arg

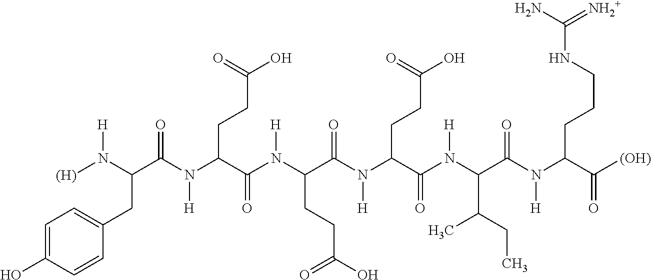

wherein the amino groups can be free or protonated and the carboxyl groups can be free or deprotonated.

Oligopeptides that are even more preferred additionally contain valine which is preferably bonded to the arginine. Hair treatment agents that are more preferred as contemplated herein are therefore characterized in that the oligopeptide contained in the hair treatment agent comprises at least one amino acid sequence Tyr-Glu-Glu-Glu-Ile-Arg-Val

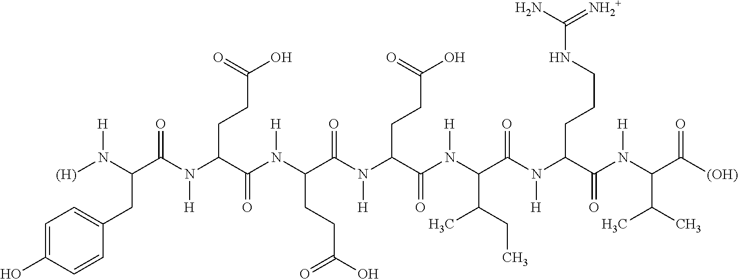

wherein the amino groups can be free or protonated and the carboxyl group can be free or deprotonated.

Oligopeptides that are even more preferred additionally contain leucine which is preferably bonded to the valine. Hair treatment agents that are more preferred as contemplated herein are therefore characterized in that the oligopeptide contained in the hair treatment agent comprises at least one amino acid sequence Tyr-Glu-Glu-Glu-Ile-Arg-Val-Leu

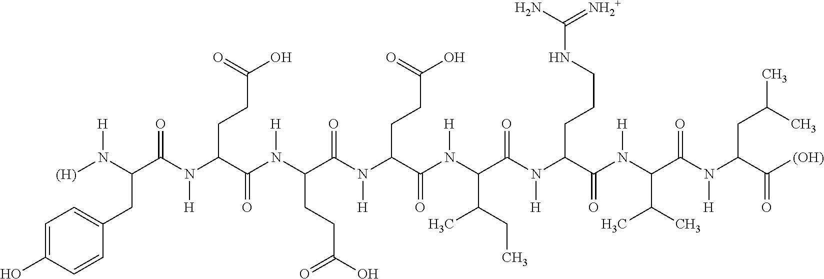

wherein the amino groups can be free or protonated and the carboxyl groups can be free or deprotonated.

Particularly preferred oligopeptides additionally contain leucine which is preferably bonded to the tyrosine. Hair treatment agents that are more preferred as contemplated herein are therefore characterized in that the oligopeptide contained in the hair treatment agent comprises at least one amino acid sequence Leu-Tyr-Glu-Glu-Glu-Ile-Arg-Val-Leu

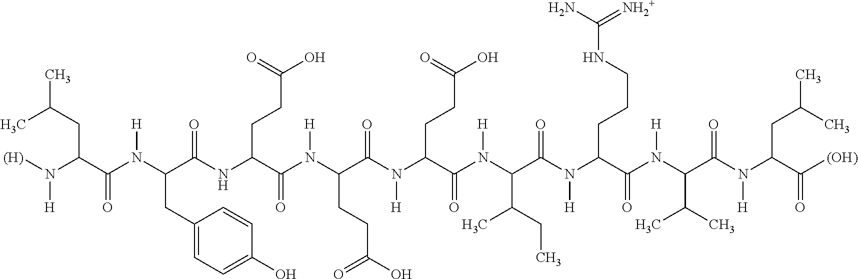

wherein the amino groups can be free or protonated and the carboxyl groups can be free or deprotonated.

Agents as contemplated herein very particularly preferably contain at least two oligopeptides which meet the above-mentioned criteria, but are different from one another. By way of example, the use of hair treatment agents that contain at least two different oligopeptides A and B, which both contain the amino acid sequence Glu-Glu-Glu, is preferred.

Such oligopeptides A and B different from one another correspond to one another in that they carry three successive Glu amino acids, but have differences in the amino acids bonded thereahead or thereafter. Different peptides which match in part to an extent greater than in the above-mentioned three amino acids are preferred.

Hair treatment agents that are more preferred are thus characterized in that at least two different oligopeptides A and B which both contain the amino acid sequence Glu-Glu-Glu-Ile are contained in the hair treatment agent.

Hair treatment agents which contain at least two different oligopeptides A and B which both contain the amino acid sequence Tyr-Glu-Glu-Glu are also preferred.

Hair treatment agents that are even more preferred are characterized in that they contain at least two different oligopeptides A and B which both contain the amino acid sequence Glu-Glu-Glu-Ile-Arg.

Hair treatment agents that are also even more preferred are characterized in that the hair treatment agent contains at least two different oligopeptides A and B which both contain the amino acid sequence Tyr-Glu-Glu-Glu-Ile.

Hair treatment agents that are preferred as contemplated herein are therefore characterized in that the oligopeptide comprises at least one amino acid sequence Tyr-Glu-Glu-Glu-Ile

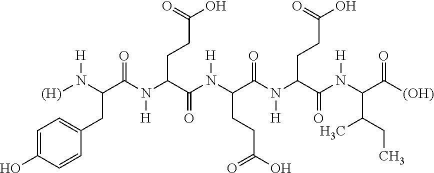

wherein the amino group can be free or protonated and the carboxyl group can be free or deprotonated.

Hair treatment agents that are very particularly preferred are characterized in that the hair treatment agent contains at least two different oligopeptides A and B which both contain the amino acid sequence Glu-Glu-Glu-Ile-Arg.

Hair treatment agents that are also very particularly preferred are characterized in that the hair treatment agent contains at least two different oligopeptides A and B which both contain the amino acid sequence Tyr-Glu-Glu-Glu-Ile-Arg.

There is preferably an even greater structural conformity in the oligopeptides. Hair treatment agents that contain at least two different oligopeptides A and B which both contain the amino acid sequence Glu-Glu-Glu-Ile-Arg-Val are thus further preferred embodiments of the present invention.

Embodiments that are also preferred are hair treatment agents that contain at least two different oligopeptides A and B which both contain the amino acid sequence Tyr-Glu-Glu-Glu-Ile-Arg-Val.

Hair treatment agents as contemplated herein that are even more preferred are characterized in that they contain at least two different oligopeptides A and B which both contain the amino acid sequence Glu-Glu-Glu-Ile-Arg-Val-Leu.

Hair treatment agents as contemplated herein that are also even more preferred are characterized in that they contain at least two different oligopeptides A and B which both contain the amino acid sequence Tyr-Glu-Glu-Glu-Ile-Arg-Val-Leu.

Hair treatment agents that are preferred as contemplated herein are therefore characterized in that the oligopeptide comprises at least one amino acid sequence Tyr-Glu-Glu-Glu-Ile-Arg-Val-Leu

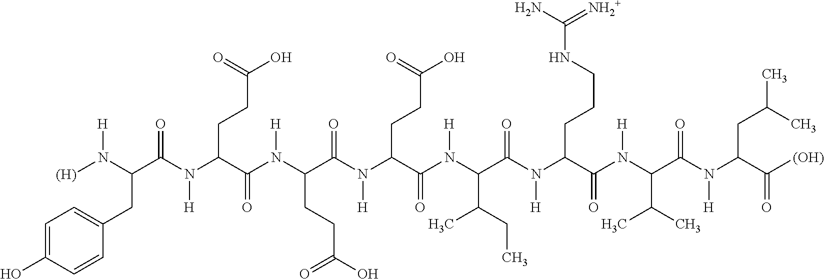

wherein the amino groups can be free or protonated and the carboxyl groups can be free or deprotonated.

Hair treatment agents as contemplated herein that are particularly preferred are characterized in that they contain at least two different oligopeptides A and B, wherein the oligopeptide A comprises the amino acid sequence Leu-Tyr-Glu-Glu-Glu-Ile-Arg-Val-Leu

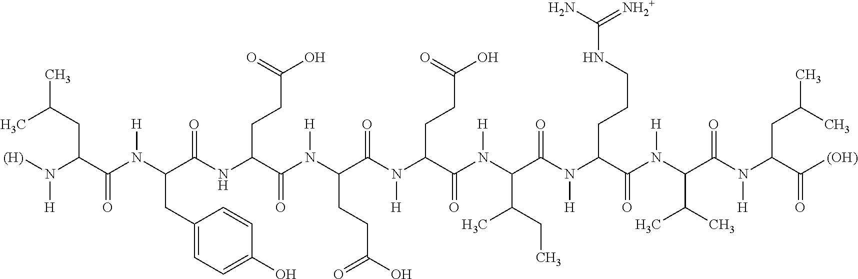

wherein the amino groups can be free or protonated and the carboxyl groups can be free or deprotonated
and the oligopeptide B comprises the amino acid sequence Tyr-Glu-Glu-Glu-Ile-Arg-Val-Leu

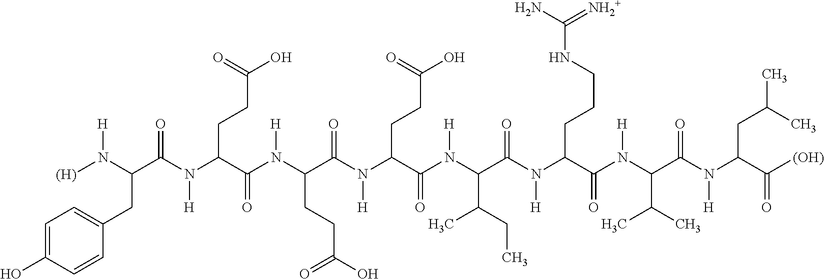

wherein the amino groups can be free or protonated and the carboxyl groups can be free or deprotonated.

In very particularly preferred hair treatment agents of this last embodiment, these agents contain—in relation to the weight of the agent—about 0.00001 to about 1% by weight oligopeptide A and about 0.00001 to about 1% by weight oligopeptide B.

In hair treatment agents of this last embodiment that are more preferred, these agents contain—in relation to the weight of the agent—about 0.00005 to about 0.1% by weight oligopeptide A and about 0.00005 to about 0.1% by weight oligopeptide B.

In hair treatment agents of this last embodiment that are even more preferred, these agents contain—in relation to the weight of the agent—about 0.0001 to about 0.01% by weight oligopeptide A and about 0.0001 to about 0.001% by weight oligopeptide B.

In embodiments, the oligopeptides which meet the above-mentioned requirements can be obtained advantageously from keratinic materials. It is preferred as contemplated herein for these oligopeptides to be used in high proportions in relation to the total keratinic peptide content of the agents.

It is very particularly preferred that the highest proportion possible of all keratinic peptides contained in the agent as contemplated herein satisfies the above-mentioned conditions.

Preferred hair treatment agents as contemplated herein are characterized in that at least at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides contained in the agent comprise the amino acid sequence Glu-Glu-Glu.

Hair treatment agents as contemplated herein that are more preferred are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides contained in the agent comprise the amino acid sequence Glu-Glu-Glu-Ile.

Hair treatment agents as contemplated herein that are even more preferred are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides contained in the agent comprise the amino acid sequence Tyr-Glu-Glu-Glu.

Hair treatment agents as contemplated herein that are particularly preferred are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides contained in the agent comprise the amino acid sequence Tyr-Glu-Glu-Glu-Ile.

Hair treatment agents as contemplated herein that are very particularly preferred are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides contained in the agent comprise the amino acid sequence Tyr-Glu-Glu-Glu-Ile-Arg.

Hair treatment agents as contemplated herein that are even more preferred are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides contained in the agent comprise the amino acid sequence Tyr-Glu-Glu-Glu-Ile-Arg-Val.

Hair treatment agents as contemplated herein that are preferred in particular are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides contained in the agent comprise the amino acid sequence Tyr-Glu-Glu-Glu-Ile-Arg-Val-Leu.

The above-mentioned conditions relate to the total content in the agents as contemplated herein of peptides originating from keratinic materials. In addition to the oligopeptide keratinic origin, further peptides and/or protein hydrolyzates can of course be used, for example from other native sources. By way of example, the additional use of wheat protein hydrolyzates is preferred.

Vitamins are preferably understood to mean the following vitamins, provitamins and vitamin precursors and derivatives thereof:
Vitamin A: the group of substances referred to as vitamin A includes retinol (Vitamin A₁) and 3,4-didehydroretinol (Vitamin A₂). β-carotin is the provitamin of retinol. Examples of suitable vitamin A components include vitamin A acid and esters thereof, vitamin A aldehyde, and vitamin A alcohol as well as esters thereof, such as the palmitate and acetate.
Vitamin B: the vitamin B group or the vitamin B complex includes, inter alia,
   Vitamin B₁ (thiamin)
   Vitamin B₂ (riboflavin)
   Vitamin B₃. This often includes the compounds of nicotinic acid and nicotinic acid amide (niacinamide).
   Vitamin B₅ (pantothenic acid and panthenol). In the context of this group, panthenol is preferably used. Useable derivatives of panthenol are especially the esters and ethers of panthenol, pantolactone, and also cationically derivatized panthenols. Specific representatives are, for example, panthenol triacetate, panthenol monoethyl ether and monoacetate thereof, as well as cationic panthenol derivatives.
   Vitamin B₆ (pyridoxine and also pyridoxamine and pyridoxal).
Vitamin C (ascorbic acid): the use in the form of the palmitic acid ester, the glucosides, or phosphates can be preferred. The use in combination with tocopherols can also be preferred.
Vitamin E (tocopherols, in particular α-tocopherol).
Vitamin F: the term “vitamin F” is usually understood to mean essential fatty acids, in particular linoleic acid, linolenic acid and arachidonic acid.
Vitamin H: The compound (3aS,4S,6aR)-2-oxohexahydrothienol[3,4-d]-imidazol-4-valeric acid denotes vitamin H, for which the trivial name however (biotin) has become accepted.

Vitamins, provitamins and vitamin precursors from the groups A, B, E and H are particularly preferred. Nicotinic acid amide, biotin, pantolactone and/or panthenol are preferred in particular. The weight share of the vitamin(s), vitamin derivative(s), and/or the vitamin precursor(s) in the total weight of the hair treatment agent is preferably about 0.001 to about 2% by weight, particularly preferably about 0.005 to about 1% by weight, and in particular about 0.01 to about 0.5% by weight.

Suitable plant extracts are understood to be extracts which can be produced from all parts of a plant. These extracts are usually produced by extraction of the entire plant. However, in individual cases it may also be preferred to produce the extracts exclusively from flowers and/or leaves of the plant. In particular, the extracts from green tea, oak bark, stinging nettle, hamamelis, hops, henna, camomile, burdock root, field horsetail, hawthorn, linden flowers, lychee, almonds, aloe vera, spruce needles, horse chestnut, sandal wood, juniper, coconut, mango, apricot, lime, wheat, kiwi, melon, orange, grapefruit, sage, rosemary, birch, malva, lady's smock, broad-leaved thyme, common yarrow, thyme, lemon balm, rest-harrow, coltsfoot, marshmallow, ginseng, ginger root, echinacea purpurea, olive, boerhavia diffusa root, fennel and celery are suitable.

Extracts from green tea, stinging nettle, hamamelis, chamomile, aloe vera, ginseng, echinacea purpurea, olive and/or boerhavia diffusa root are particularly preferred for use in the compositions as contemplated herein.

Water, alcohols, and mixtures thereof can be used as extracting agents for producing the specified plant extracts. Here, lower alcohols such as ethanol and isopropanol, but in particular polyvalent alcohols such as ethylene glycol and propylene glycol, both as sole extracting agent and mixed with water, are preferred alcohols. Plant extracts based on water/propylene glycol in a ratio of from 1:10 to 10:1 have proven to be particularly suitable.

The plant extracts can be used both in pure and diluted form. If used in diluted form, they usually contain approximately 2-80% by weight of active substance and, as solvent, the extracting agent or extracting agent mixture used for their recovery.

The plant extracts can be used in the hair treatment agents as contemplated herein (in relation to the total weight of the agents) preferably in an amount of from about 0.01 to about 10% by weight, more preferably from about 0.05 to about 7.5% by weight, and in particular from about 0.1 to about 5% by weight.

Glycerol can be added to the hair cleansing and nourishing agents separately in an amount of up to about 10% by weight (in relation to the total weight of the agent). However, it can also be part of the aforementioned aqueous-alcoholic carrier.

It has been found that the hair treatment agents as contemplated herein are also suitable for use as an anti-dandruff preparation.

The total weight of anti-dandruff agents in the total weight of the hair treatment agent can be preferably about 0.01 to about 10% by weight, more preferably about 0.025 to about 7.5% by weight, particularly preferably about 0.05 to about 5% by weight, and in particular about 0.075 to about 3% by weight.

Suitable anti-dandruff active substances can be selected from piroctone olamines, climbazole, zinc pyrithione, ketoconazoles, salicylic acid, sulfur, selenium sulfide, tea preparations, undecenoic acid derivatives, burdock extracts, poplar extracts, stinging nettle extracts, walnut shell extracts, birch extracts, willow bark extracts, rosemary extracts and/or arnica extracts.

Climbazole, zinc pyrithione, and piroctone olamines are preferred.

Further active substances, auxiliaries and additives that can be contained with preference in the hair treatment agents as contemplated herein are, for example,
humectants,
fragrances,
UV filters,
thickening agents such as gelatins or plant gums, for example agar-agar, guar gum, alginates, xanthan gum, gum arabic, karaya gum, locust bean flour, linseed gums, dextrans, cellulose derivatives, for example methyl cellulose, hydroxyalkyl cellulose and carboxymethyl cellulose, starch fractions and derivatives, such as amylose, amylopectin and dextrins, clays and sheet silicates, such as bentonite or fully synthetic hydrocolloids such as polyvinyl alcohol, the Ca, Mg or Zn soaps,
structurants, such as maleic acid and lactic acid,
dimethyl isosorbide,
cyclodextrins,
fiber structure-improving active substances, in particular mono-, di- and oligosaccharides, such as glucose, galactose, fructose, fruit sugar, and lactose,
dyes for coloring the agent,
active substances such as bisabolol and/or allantoin,
complexing agents, such as EDTA, NTA, β-alanine diacetic acid and phosphonic acids,
ceramides. Ceramides are understood to mean N-acylsphingosine (fatty acid amides of sphingosine) or synthetic analogues such as lipids (what are known as pseudo ceramides),
propellants, such as propane-butane mixtures, N₂O, dimethylether, CO₂ and air,
antioxidants,
additional viscosity regulators, such as salts (NaCl).

The agents as contemplated herein are preferably what are known as rinse-off products, i.e. they are rinsed out of the hair again after a certain reaction time. This reaction time is preferably less than an hour, i.e. the consumer preferably does not leave the products in the hair until the next time the hair is washed.

A further subject of the present invention is therefore a method for treating hair, in which an agent as contemplated herein is applied to dry or wet hair, is left there for a period of from about 30 to about 300 seconds, and is then rinsed out.

The agents as contemplated herein lead to a significantly increased stability of artificial dyes with respect to the washing out of the color. Chemically dyed hair can thus be washed much more frequently with the agents as contemplated herein, without this resulting in an undesirable bleeding or fading of the color.

A further subject as contemplated herein is therefore the use of agents as contemplated herein for reducing the washout of color from chemically dyed hair.

That stated with regard to the agents as contemplated herein applies, mutatis mutandis, in respect of preferred embodiments of the method as contemplated herein and the use as contemplated herein.

### Examples

### All Values in % by Weight

Hair shampoos series 1

| | 1 | 2 | 3 | 4 | 5 | 6 |
|---|---|---|---|---|---|---|
| Sodium laureth sulfate (AS) | 10.0 | 10.0 | 10.0 | 10.0 | 10.0 | 10.0 |
| Cocoamidopropyl betaine | 4.0 | 4.0 | 4.0 | 4.0 | 4.0 | 4.0 |
| (AS) | | | | | | |
| Ammonium lauryl sulfate | 4.0 | 4.0 | 4.0 | 4.0 | 4.0 | 4.0 |
| Polyquaternium-10 | 0.3 | 0.3 | 0.3 | 0.3 | 0.3 | 0.3 |
| Cocamide MEA (AS) | 0.8 | 0.8 | 0.8 | 0.8 | 0.8 | 0.8 |
| PEG-7 glyceryl cocoate | 1.0 | 1.0 | 1.0 | 1.0 | 1.0 | 1.0 |
| Nicotinic acid amide | 0.3 | 0.3 | 0.3 | 0.3 | 0.3 | 0.3 |
| Lactic acid | 0.2 | 0.2 | 0.2 | 0.2 | 0.2 | 0.2 |
| Sunflower oil | 1.0 | 1.0 | 1.0 | 1.0 | 1.0 | 1.0 |
| Panthenol | 0.5 | 0.5 | 0.5 | 0.5 | 0.5 | 0.5 |
| Laureth-2 | 1.2 | 1.2 | 1.2 | 1.2 | 1.2 | 1.2 |
| Sodium chloride | 1.3 | 1.3 | 1.3 | 1.3 | 1.3 | 1.3 |
| o-phenylphenol | 0.2 | 0.2 | 0.2 | 0.2 | 0.2 | 0.2 |
| Ethyl lauroyl arginate | — | 0.4 | 0.3 | 0.2 | 0.1 | 0.05 |
| Glutaraldehyde | — | 0.05 | 0.06 | 0.07 | 0.08 | 0.1 |
| Dimethyloxazolidine | — | 0.05 | 0.06 | 0.07 | 0.08 | 0.1 |
| Phenoxyethanol | — | 0.05 | 0.05 | 0.05 | 0.05 | 0.05 |
| Phenoxyisopropanol | — | 1.0 | 0.5 | 0.25 | 0.2 | 0.1 |
| o-cymen-5-ol | — | 0.1 | 0.05 | 0.1 | 0.05 | 0.1 |
| Hexetidine | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Fragrance | 0.1 | 0.1 | 0.1 | 0.1 | 0.1 | 0.1 |

| | |
|---|---|
| Water | to 100 |

Hair shampoos series 2

| | 1 | 2 | 3 | 4 | 5 | 6 |
|---|---|---|---|---|---|---|
| Sodium laureth sulfate (AS) | 7.0 | 7.0 | 7.0 | 7.0 | 7.0 | 7.0 |
| Disodium | 4.0 | 4.0 | 4.0 | 4.0 | 4.0 | 4.0 |
| cocoamphodiacetate (AS) | | | | | | |
| Coco glucoside | 3.0 | 3.0 | 3.0 | 3.0 | 3.0 | 3.0 |
| Cocamide MEA (AS) | 0.3 | 0.3 | 0.3 | 0.3 | 0.3 | 0.3 |
| Guar hydroxypropyl | 0.1 | 0.1 | 0.1 | 0.1 | 0.1 | 0.1 |
| trimoniumchloride | | | | | | |
| Panthenol | 0.2 | 0.2 | 0.2 | 0.2 | 0.2 | 0.2 |
| Jojoba seed oil | 0.05 | 0.05 | 0.05 | 0.05 | 0.05 | 0.05 |
| Wheat protein hydrolyzate | 0.3 | 0.3 | 0.3 | 0.3 | 0.3 | 0.3 |
| Citric acid | 0.4 | 0.4 | 0.4 | 0.4 | 0.4 | 0.4 |
| PEG-120 methyl | 0.7 | 0.7 | 0.7 | 0.7 | 0.7 | 0.7 |
| glucose dioleate | | | | | | |
| Sodium-o-phenylphenolate | 0.2 | 0.2 | 0.2 | 0.2 | 0.2 | 0.2 |
| Ethyl lauroyl arginate | — | 0.4 | 0.3 | 0.2 | 0.1 | 0.05 |
| Glutaraldehyde | — | 0.05 | 0.06 | 0.07 | 0.08 | 0.1 |
| Dimethyloxazolidine | — | 0.05 | 0.06 | 0.07 | 0.08 | 0.1 |
| Phenoxyethanol | — | 0.05 | 0.05 | 0.05 | 0.05 | 0.05 |
| Phenoxyisopropanol | — | 1.0 | 0.5 | 0.25 | 0.2 | 0.1 |
| o-cymen-5-ol | — | 0.1 | 0.05 | 0.1 | 0.05 | 0.1 |
| Hexetidine | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Fragrance | 0.1 | 0.1 | 0.1 | 0.1 | 0.1 | 0.1 |

| | |
|---|---|
| Water | to 100 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment. It being understood that various changes may be made in the function and arrangement of elements described in an exemplary embodiment without departing from the scope as set forth in the appended claims.

1. A hair treatment agent comprising:
a) at least one anionic surfactant,
b) at least one amphoteric and/or non-ionic surfactant,
c) at least one cationic polysaccharide polymer,
d) o-phenylphenol and/or salts of o-phenylphenol 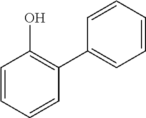 [SMILES: Oc1ccccc1-c1ccccc1]
e) at least one organic acid, and
f) at least one lipid component.

2. The hair treatment agent as claimed in claim 1, comprising, based on the weight of the agent, about 0.5 to about 20% by weight of a) anionic surfactant(s).

3. The hair treatment agent as claimed in claim 1, comprising, based on the weight of the agent, about 0.3 to about 10% by weight of b) amphoteric surfactant(s).

4. The hair treatment agent as claimed in claim 1, comprising, based on the weight of the agent, about 0.3 to about 10% by weight of b) non-ionic surfactant(s).

5. The hair treatment agent as claimed in claim 1, comprising as cationic polysaccharide polymer(s) c) at least one polymer from the group of cationic cellulose polymers and/or cationic guar derivatives.

6. The hair treatment agent as claimed in claim 1, comprising, based on the weight of the agent, about 0.001 to about 0.4% by weight d) o-phenylphenol.

7. The hair treatment agent as claimed in claim 1, comprising, based on the weight of the agent, about 0.001 to about 0.4% by weight of d) sodium orthophenylphenolate.

8. The hair treatment agent according to claim 1, comprising e) organic acid(s) chosen from the group of formic acid, oxalic acid, maleic acid, lactic acid, tartaric acid, or citric acid.

9. The hair treatment agent as claimed in claim 1, comprising, based on the weight of the agent, about 0.01 to about 5% by weight of f) lipid component(s).

10. The hair treatment agent as claimed in claim 1, wherein the f) lipid component(s) is/are chosen from triglyceride fats, mono- and diglycerides of saturated, linear C12-C40 fatty acids, natural and synthetic waxes, fatty alcohols and wax alcohols, C10-C40 fatty acids, esters of fatty alcohols and fatty acids that are not liquid at 25° C., fatty acid amides, and any mixtures of these substances.

11. The hair treatment agent as claimed in claim 1, comprising, based on the total weight of the agent, about 0.01 to about 3% by weight of c) cationic polysaccharide polymer(s).

12. The hair treatment agent according to claim 8, comprising, based on the weight of the agent, about 0.001 to about 10% by weight of e) organic acid(s) chosen from the group of formic acid, oxalic acid, maleic acid, lactic acid, tartaric acid, or citric acid.

13. A method for treating hair, comprising:
applying a hair treatment agent to dry or moist hair, wherein the hair treatment agent comprises:
a) at least one anionic surfactant,
b) at least one amphoteric and/or non-ionic surfactant,
c) at least one cationic polysaccharide polymer,
d) o-phenylphenol and/or salts of o-phenylphenol 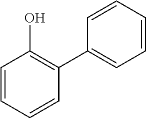 [SMILES: Oc1ccccc1-c1ccccc1]
e) at least one organic acid, and
f) at least one lipid component,
where the hair treatment is agent is left on the hair for a period of about 30 to about 300 seconds and then rinsed off.

14. The hair treatment agent according to claim 1, comprising, based on the weight of the agent:
about 0.5 to about 20% by weight of a) anionic surfactant(s);
about 0.3 to about 10% by weight of b) amphoteric surfactant(s) or/or non-ionic surfactant(s);
about 0.01 to about 3% by weight of c) cationic polysaccharide polymer(s);
about 0.001 to about 0.4% by weight of d) o-phenylphenol;
about 0.001 to about 10% by weight of e) organic acid(s); and
about 0.01 to about 5% by weight of f) lipid component(s).

15. The hair treatment agent according to claim 14, comprising:
as c) cationic polysaccharide polymer(s) at least one polymer from the group of cationic cellulose polymers and/or cationic guar derivatives;
sodium orthophenylphenolate as the d) o-phenylphenol;
e) organic acid(s) chosen from the group of formic acid, oxalic acid, maleic acid, lactic acid, tartaric acid, or citric acid; and
f) lipid component(s) chosen from triglyceride fats, mono- and diglycerides of saturated, linear C12-C40 fatty acids, natural and synthetic waxes, fatty alcohols and wax alcohols, C10-C40 fatty acids, esters of fatty alcohols and fatty acids that are not liquid at 25° C., fatty acid amides, and any mixtures of these substances.
